(19) United States
(12) Patent Application Publication
(10) Pub. No.: US 2015/0088004 A1
(43) Pub. Date: Mar. 26, 2015

(54) METHOD FOR MONITORING AN ACCURATE HEART RATE

(71) Applicant: BIORICS NV, Heverlee (BE)

(72) Inventors: Daniel Berckmans, Kessel-Lo (BE); Vasileios Exadaktylos, Thessaloniki (GR); Jean-Marie Aerts, Herent (BE); Joachim Taelman, Herent (BE)

(21) Appl. No.: 14/388,920

(22) PCT Filed: Mar. 15, 2013
(86) PCT No.: PCT/EP2013/055494

§ 371 Date: Sep. 29, 2014

(30) Foreign Application Priority Data
Mar. 28, 2012 (GB) 1205472.2

(51) Int. Cl. A61B 5/0205; A61B 5/0476; A61B 5/00; A61B 5/11

(52) U.S. Cl. CPC A61B 5/0205; A61B 5/1118; A61B 5/1112; A61B 5/0476; A61B 5/1102; A61B 5/7221; A61B 5/7278; A61B 5/02433
USPC 600/483

(57) ABSTRACT

A method for monitoring a more accurate heart rate of a human or an animal, wherein at least one heart rate or electrocardiogram (ECG) signal and at least one activity signal is measured and wherein, when said measured heart rate or ECG signal is of low quality, the heart rate or ECG signal is at least partially rejected and replaced by a simulated heart rate or ECG signal, which is calculated from a predetermined relationship between the activity signal and the heart rate or ECG signal. By applying this method in real time using on-line modelling a predetermined relationship is continuously updated to have an accurate modelled heart rate.

The present invention concerns a method for monitoring a heart rate of a human or an animal, wherein at least one heart rate signal and at least one activity signal is measured for a human or an animal.

The activity signal is intended to be a measure for the level of aerobic metabolic activity and/or mental activity.

The heart rate signal is intended to be a signal from which the heart rate of the human or animal can be obtained independent of external conditions and independent of the mental or physical conditions of the human or animal. Examples of suitable heart rate signals are electrical signals measured from the body of humans and/or animals, electrocardiogram (ECG) signals, ballistocardiogram (BCG) signals, blood pressure signals, infrared camera signals.

There are many applications were monitoring of heart rate obtained from heart rate signals are creating added value. Several systems are available to monitor the heart rate of humans and animals, e.g. horses.

When the heart muscle is active, it produces an electrical signal that can be measured on the body, directly, via e.g. an ECG signal or also, indirectly, via e.g. interference of heart rate signals with other electrical measurements on the body such as an electromyogram (EMG). The ECG or heart rate measurements start by measuring the electrical potential difference over a number of positions on the body. The minimum number of positions is two. This means that at least one sensor has to measure the electrical signal on the skin either by making contact with the skin or not. This can be done by stickers or by wearing a belt that has at least two contact points with electrical conductance on the skin. Alternatively, sensors positioned in the direct environment of the user, like in a car seat or in clothes can also be used. The heart rate or ECG signal may also be obtained from capacitive sensors, which do not need to make a physical contact with the skin of a human or an animal.

The problem with e.g. stickers is that they are uncomfortable to be used for sports or every day applications since they are unpractical and time consuming to be positioned on the body. Moreover they are irritating the skin when used for some time.

A chest belt with sensors is accepted by many sportspeople during their sports activity, but it still takes special attention and care to use it during normal training activity. It would be handier to integrate the required electrodes into shirts as is done today by several producers of smart textiles.

The problem with all known solutions, such as e.g. belts and shirts, intelligent textiles or smart fabrics, is that there is not always a good interaction or electrical contact between on one side the sticker, the belt or shirt and on the other side the skin. All sensors that are in contact with the skin or that are intended to be located in the direct vicinity of the body are moving at moments of high activity like e.g. a sprint when doing active movements like for example running or biking or jumping in other sports or intensive movements like in tennis, rugby, volleyball, etc. Another cause of a less optimal interaction is the influence of sweating on the electrical contact. Hence, the interaction between different sensors and the body or skin is not always optimal for obtaining a good heart rate signal.

As a consequence no good measurement of heart rate is realized during certain periods of the performed activities. It can be shown that, depending on the type of sensor up to 55% of heart rate signals cannot be measured in a reliable way during a normal soccer training.

The main function of the heart muscle is transport of blood and oxygen throughout the body of a human or an animal. As such the heart can be seen as a pump. As a consequence, the heart rate can also be obtained from heart rate signals other than electrical measurements on the body. These heart rate signals include, amongst others, a ballistocardiogram, which reflects changes in force and pressure due to fluid mechanical properties of flooding blood, and infrared camera signals, which reflect changes in blood oxygenation due to pulsing properties of the heart as blood pump.

The invention aims to remedy the above mentioned disadvantages of the measuring systems of the heart rate signals by suggesting a simple solution with respect to a method for monitoring a heart rate.

The above mentioned objects are realised by the method and device having the specific features set out in the appended claims. Specific features for preferred embodiments of the invention are set out in the dependent claims.

Practically, in the method, according to the invention, the heart rate signal or a heart rate obtained from the heart rate signal is at least partially rejected when said measured heart rate signal is of low quality, and a rejected heart rate or a rejected heart rate signal is replaced by a simulated heart rate or a simulated heart rate signal, which is obtained from a predetermined relationship between the activity signal and the heart rate or the heart rate signal.

By applying the method in real time using on-line modelling the predetermined relationship is preferably continuously updated to have an accurate modelled heart rate.

Other particularities and advantages of the invention will become clear from the following description and accompanying drawings of practical embodiments of the method of the invention; the description and drawings are given as an example only and do not limit the scope of the claimed protection in any way.

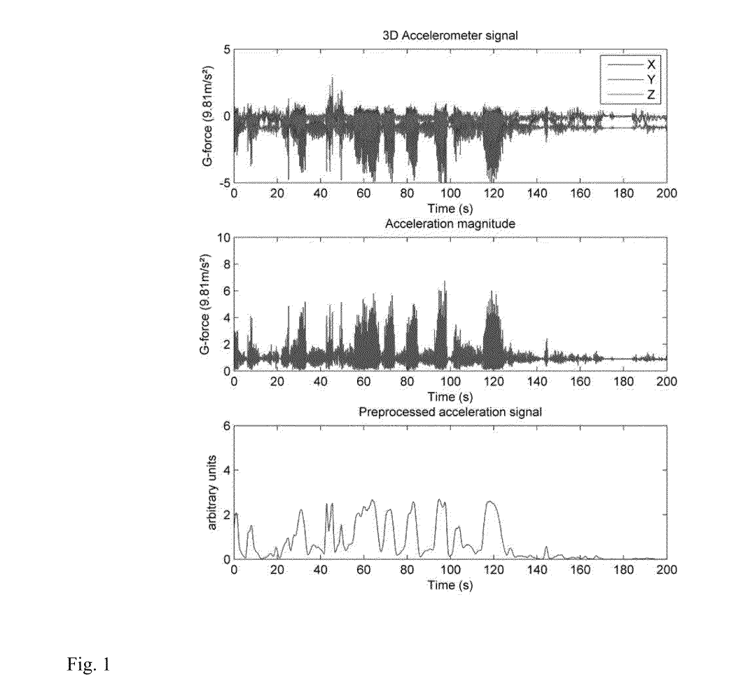

FIG. 1 is a representation of typical signals obtained from a 3D accelerometer attached to a body. The first graph represents a 3D accelerometer signal in the X, Y and Z direction. The second graph represents the acceleration magnitude vector and the third graph represents a signal derived from the original signals that can be used as activity vector.

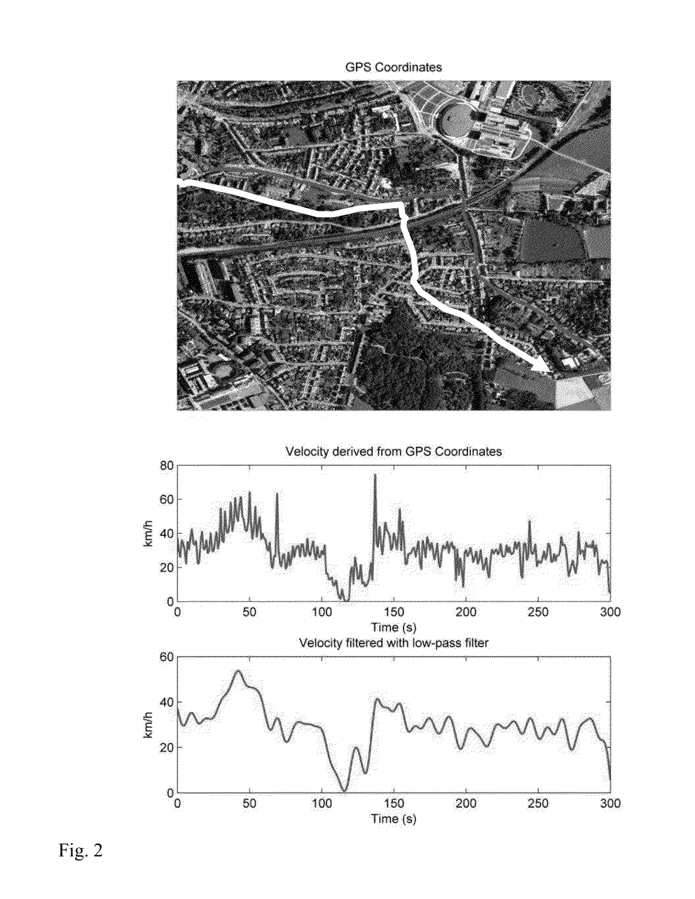

FIG. 2 is a representation of a global positioning system (GPS) signal from which an activity signal can be derived such as a velocity signal. The first graph is a representation of mapped longitude and latitude coordinates of a GPS signal. The second graph is the velocity signal as a function of time derived from the GPS signal. The third graph is a processed velocity signal that is obtained from the velocity signal of the second graph.

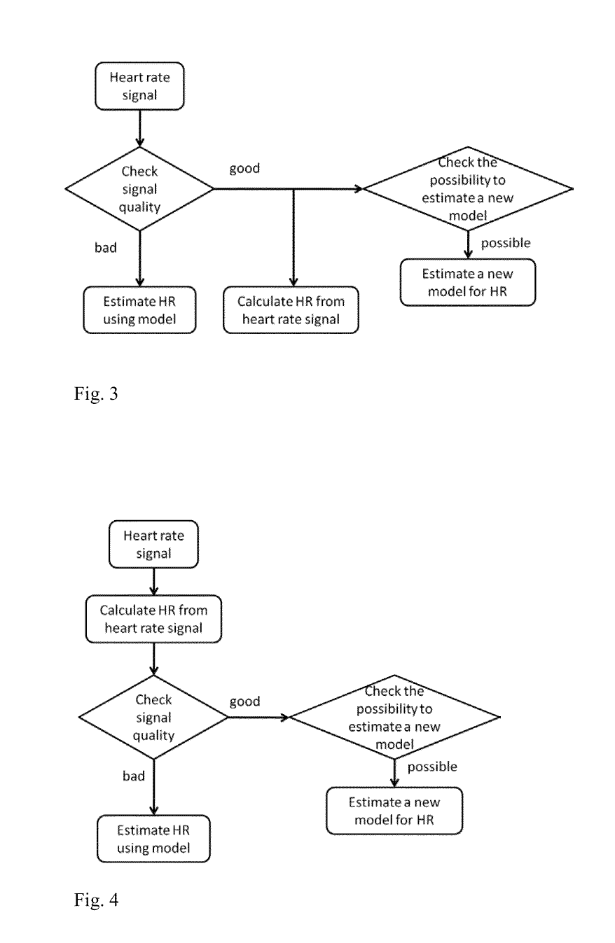

FIG. 3 is a flow chart of a method according to the invention in which the quality of the measured heart rate signal is checked.

FIG. 4 is a flow chart of a method according to the invention in which the quality of the heart rate obtained from the measured heart rate signal is checked.

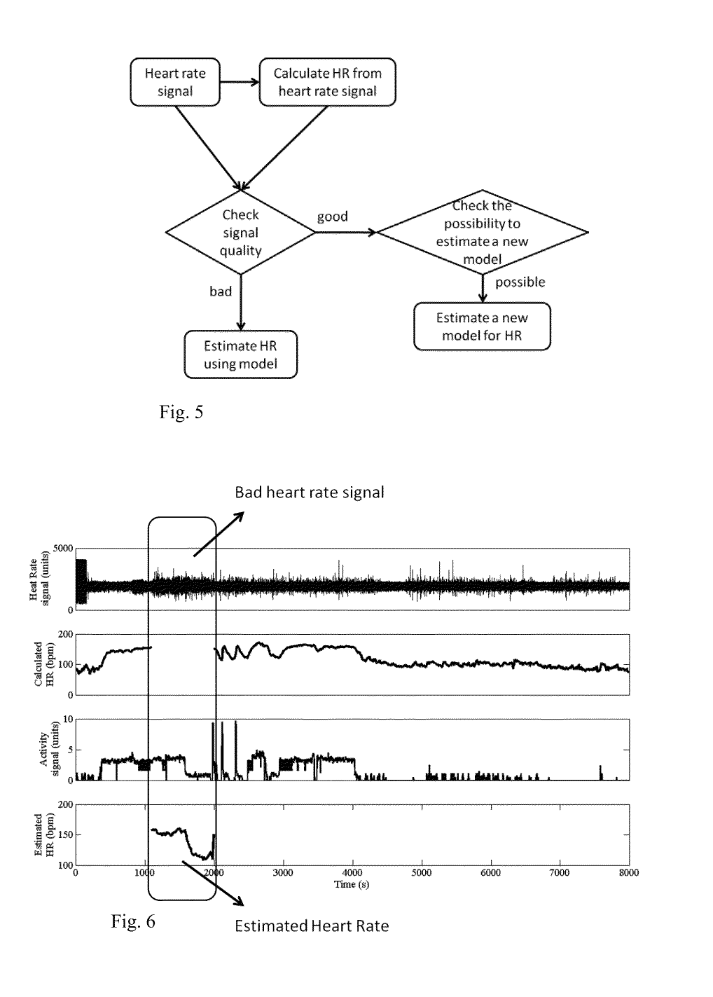

FIG. 5 is a flow chart of a method according to the invention in which the quality of both the measured heart rate signal and the heart rate obtained therefrom is checked.

FIG. 6 is a graphical representation of a measured heart rate signal, a calculated heart rate obtained from the measured heart rate signal, a measured activity signal and an estimated heart rate obtained from the activity signal based on the relationship between the heart rate signal and/or the heart rate and the activity signal, according to a method of the invention.

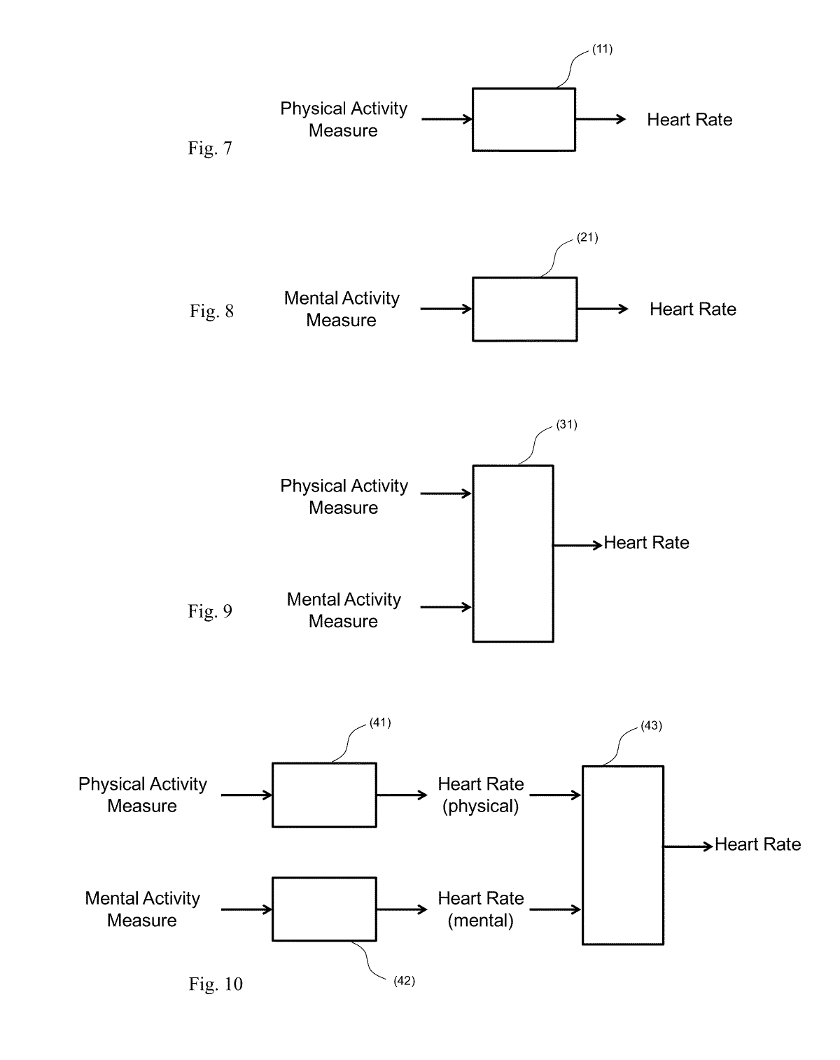

FIG. 7 schematically represents the relation between the physical activity and the heart rate (HR).

FIG. 8 schematically represents the relation between the mental activity and the heart rate (HR).

FIG. 9 schematically represents the relation between the physical activity, the mental activity and the heart rate (HR).

FIG. 10 schematically represents the relation between the physical activity, the mental activity and the heart rate (HR) composed of a physical HR component and a mental HR component.

The invention generally concerns a method for monitoring the heart rate by measuring a heart rate signal and solves the above described problems based on the fact that:

1. Bad measurements of the a heart rate signal are occurring now and then at e.g. periods of high activity;

2. There is a relationship between the heart rate and the body activity, in particular metabolic aerobic activity, since for example the heart rate generates the energy to move the body.

The activity signal is by preference a measure for the level of aerobic metabolic activity and may be obtained from at least one activity sensor. Alternatively, the activity signal is a measure of mental activity.

The activity sensor may comprise, for example, a sensor applied to the body, a motion sensor, an accelerometer, a global positioning system (GPS) and/or a camera system. The sensor applied to the body may be used for measurement of e.g. power, pressure, oxygen consumption, respiration and respiration rate and/or brain waves. The camera system may be used for e.g. measuring body motion from a distance of the body. In another example, the activity sensor may comprise a measure of brainwaves by means of an Electro-Encephalogram (EEG) or parameters extracted from such a measurement, such as, for example, pressure of delta waves.

FIG. 1 shows typical signals from a 3D accelerometer attached to a human body while performing some activity. For each of the directions according to the X, Y and Z axes a signal is obtained. From these measured raw signals an acceleration magnitude signal can be calculated and further processed to obtain a pre-processed acceleration signal. All these signals can be used as suitable activity signals according to the present invention.

FIG. 2 shows schematically a global positioning system (GPS) signal from a GPS receiver attached to a human body while performing activity. Longitude and latitude coordinates are monitored as a function of time. From this data further activity signals can be derived such as, for example, a velocity signal as a function of time as shown in the graphs of FIG. 2. These signals can be processed, using any know technique, to derive further activity signals suitable to be used in a method according to the present invention.

The heart rate signal may be obtained from, for example, at least one set of electrodes applied to a body of a human or an animal. This signal may comprise an ECG signal.

By using some criteria for the quality of the measured heart rate or heart rate signal, it is possible to detect for what data periods the sensors deliver a good heart rate signal and/or a good heart rate measurement.

In FIG. 3 a method according to the invention is illustrated wherein the quality of the heart rate signal is checked after which the heart rate is obtained from a good heart rate signal. When the heart rate signal is of good quality and when an activity signal is measured, the relationship between heart rate and/or heart rate signal and the activity signal is estimated in a new model. When the heart rate signal is of bad quality, the heart rate is estimated from the measured activity signal by using an existing, preferably most recent, model for the relationship between the heart rate and/or the heart rate signal and the activity signal.

In FIG. 4 a method according to the invention is illustrated wherein the quality of the heart rate is checked after the heart rate is obtained from the heart rate signal. When the heart rate obtained from the heart rate signal is of bad quality, the heart rate is estimated from the measured activity signal based on the model describing the relationship between the heart rate and/or the heart rate signal and the activity signal. Preferably the model is updated when the heart rate obtained from the heart rate signal is of good quality.

In FIG. 5 a method according to the invention is illustrated wherein both the quality of the heart rate signal and the heart rate obtained therefrom is checked. If the heart rate signal or the heart rate obtained is of bad quality, then the heart rate is estimated based on the model describing the relationship between the heart rate and/or the heart rate signal and the activity signal. If both the heart rate signal and the heart rate obtained are of good quality, then the model is updated.

Possible criteria for the quality of the measured heart rate signal may be based on (i) the physiological properties of the heart rate signal, such as e.g. the skewness of the signal, the amplitude of the signal (too high or too low), the frequency content of the signal, (ii) the signal saturation, (iii) the waveform of the signal or (iv) other typical properties of the signal.

Possible criteria for the quality of the measured ECG signal may be based on e.g. the skewness or on e.g. the frequency content of the ECG signal. Hence, a possible criterion, for example for the ECG signal, may be implemented by looking at parameters of a part of the ECG signal, e.g. in a one-second window. One parameter can be the skewness of the measured ECG signal. If the skewness is higher than e.g. one, then the ECG signal could be considered to be good, otherwise the ECG signal can be rejected. The skewness can also be filtered for obtaining a smoother signal. Another parameter can be the frequency content of the ECG signal. From the frequency, we can look at the area below graph of frequencies in the range of 2 to 20Hz. If the area is below a defined threshold, e.g. 500, then the ECG signal could be considered to be good, otherwise the ECG signal can be rejected.

Possible criteria for the quality of the measured heart rate signal may be based on e.g. the variance of the heart rate signal or on physiologically non-realistic values of the heart rate or the heart rate signal. A possible criterion for the quality of the measured heart rate signal may be implemented by looking at parameters of a part of the heart rate signal or the heart rate in beats-per-minute (bpm), e.g. in a 4-second window. These parameters can be the variance of the heart rate signal. Further, a heart rate may be rejected when e.g. for humans it is outside a realistic range of 40 to 220 bpm. Hence, the heart rate signal can be considered to be of low quality when either the signal itself is not good or when the heart rate obtained from this signal is not good, e.g. is physiologically not realistic.

The measured heart rate signal or the heart rate obtained therefrom can be compared with a set of reference values in order to evaluate the quality of this heart rate signal or this heart rate. As such the set of reference values may be a range within which the measured signal or the heart rate obtained therefrom should fit in order to qualify the signal or the heart rate as not being of a low quality and hence acceptable. The set of reference values may be obtained from average values applicable to any individual. The values can also be specific for an individual based on e.g. previously obtained values for said individual.

By measuring the heart rate in the periods where the signal is good, i.e. not of low quality, it is possible to calculate the relationship between the heart rate signal and the activity level performed by the individual at that moment and in those circumstances, i.e. at the moment of measurement and in the particular circumstances at the moment of measurement, taking into account e.g. temperature, heat losses, etc.

By using some way of activity sensor, for example an accelerometer, in combination with the heart rate measurement, a real-time relationship can be calculated between measured activity and heart rate, obtained from the heart rate signals in the “good data parts”, where the heart rate and/or the heart rate signals are rated to be of good quality, as decided by e.g. the above described conditions. When the heart rate signal is found to be of low quality, this relationship between activity level and heart rate is used in the “bad data parts” to estimate the heart rate signal from the measured activity levels, as illustrated in FIG. 6. This relationship can be defined for example in terms of a mathematical model, e.g. an autoregressive exogenous (ARX) model.

Since the relationship between activity level and heart rate is not only individually different, but also varying with, for example, the physical condition of a same individual, this combination of measurements of ECG and/or heart rate and activity level on the one side with the modelling or calculating of the relationship with heart rate in the good parts needs to be realised in real time.

This means that the method includes several steps:

Measuring a heart rate signal, such as e.g. ECG;

Measuring metabolic aerobic activity levels, using activity sensors;

Detecting continuously the good data parts by checking the quality of heart rate signals and/or heart rate measurement;

Calculating the real-time relationship between heart rate and activity level for each individual on that moment and in those circumstances;

Checking if the heart rate and/or the heart rate signal is not good enough, i.e. is of low quality, and switch then to the modelled heart rate, i.e. a heart rate and/or heart rate signal obtained from the activity measurement;

Switching back to the normal situation where the heart rate signal and/or heart rate are measured with enough quality since the measured signal is measured in a reliable way, i.e. when the measured heart rate signal and/or the heart rate obtained from the measured heart rate signal is not of low quality;

Updating the model continuously since the relationship between activity level and heart rate is depending on several variables like climate conditions, micro-environment, physical condition, health status, etc . . .

The heart rate (HR) may be the result of the above described physical activity. Hence, there is a relationship (11) between physical activity and HR as shown in FIG. 7. By estimating the relationship (11) in real-time, during the periods where the heart rate signal is of good quality, HR can be estimated based on the activity level, in particular the measured activity signal.

Additionally, the heart rate can be the result of mental activity. This includes, but is not limited to, stress, concentration, emotions, performance of a mental task, etc. In this case, a relationship (21), as shown in FIG. 8, can be found between HR and one or more measures of mental activity, e.g. power of brain waves such as alpha waves, skin conductance, body temperature, etc. This relationship can also be adapted in real-time provided that an accurate measurement of HR is available. Then, HR can be estimated from the measure of mental activity using the relationship.

HR can of course be influenced by both physical and mental tasks or activities at the same time. In this case, a relationship (31) can be estimated that links the effect of both mental and physical activity measures to HR, as shown in FIG. 9. Then the HR can be estimated using this relationship.

Alternatively, physical and mental components of HR can be separately estimated and subsequently combined to estimate the total HR, as shown in FIG. 10. More specifically, a relationship (41) between the physical activity measure and the physical component of HR can be estimated and a relationship (42) between the mental activity measure and the mental component of HR can be estimated. Subsequently, the relationship (43) between physical and mental HR components and the total HR can be estimated. The scheme that is visualised in FIG. 10 can be used to estimate HR from measurements of physical and mental activity.

Naturally, the invention is not restricted to the method according to the invention as described above. Thus, besides an accelerometer for measuring the activity of a person or animal, a global positioning system (GPS) device or a video camera may be used as well.

1. The method for monitoring a heart rate of a human or an animal, comprising the steps of
measuring at least one heart rate signal and at least one activity signal,
modeling or calculating of a predetermined relationship between the activity signal and the heart rate or a predetermined relationship between the activity signal and the heart rate signal in real time,
rejecting at least partially the measured heart rate signal or a heart rate obtained from the measured heart rate signal when said measured heart rate signal is of low quality, and
replacing a rejected heart rate or a rejected heart rate signal by a simulated heart rate or a simulated heart rate signal,
obtaining the simulated heart rate or the simulated heart rate signal from the activity signal using the predetermined relationship between the activity signal and the heart rate or the predetermined relationship between the activity signal and the heart rate signal, and
outputting the measured and/or simulated heart rate or heart rate signal.

2. The method according to claim 1, wherein the heart rate signal or the heart rate obtained from the heart rate signal is at least partially rejected by using a criterion to check the quality of the heart rate or the heart rate signal.

3. The method according to claim 1, wherein the heart rate or the heart rate signal is at least partially rejected when the heart rate or the heart rate signal deviates from a set of reference values.

4. The method according to claim 1, wherein at least one heart rate signal is measured from at least one set of electrodes applied to a body of a human or an animal.

5. The method according to claim 1, wherein the at least one heart rate signal comprise an electrocardiogram (ECG) signal, a ballistocardiogram (BCG) signal, a blood pressure signal, an infrared (IR) camera signal and/or a capacitive sensor signal.

6. The method according to claim 1, wherein a set of electrodes continuously monitor an electrocardiogram (ECG) from which said heart rate signal is obtained.

7. The method according to claim 1, wherein a capacitive sensor is used to measure said heart rate.

8. The method according to claim 1, wherein at least one activity signal is measured from at least one activity sensor applied to the body.

9. The method according to claim 1, wherein the at least one activity signal is obtained from at least one activity sensor, which comprises a motion sensor, an accelerometer, a global positioning system (GPS) device and/or a camera.

10. The method according to claim 1, wherein the activity signal comprise a power signal, a pressure signal, an oxygen consumption signal, a respiration rate, brain waves and/or GPS positions.

11. The method according to claim 1, wherein the activity signal is measured as a measure for the level of aerobic metabolic activity.

12. The method according to claim 1, wherein at least one activity signal is measured as a measure for the level of physical activity.

13. The method according to claim 1, wherein at least one activity signal is measured as a measure for the level of mental activity.

14. The method according to claim 1, further comprising continuously updating said relationship between said activity signal and said heart rate or said heart rate signal.

15. The method according to claim 1, further comprising continuously calculating the relationship between said activity signal and said heart rate or said heart rate signal in order to determine and monitor said predetermined relationship between the activity signal and the heart rate or the heart rate signal.

16. The method according to claim 1, further comprising calculating or updating said relationship between said activity signal and said heart rate or said heart rate signal dependent on external variables including climate conditions, micro-environment, physical condition, health status.

17. The method according to claim 1, further comprising sending the heart rate signal and the activity signal to a remote data processing and computing unit.

18. The method according to claim 1, further comprising:
attaching at least one sensor to a body of the human or the animal for measuring the heart rate signal;
measuring the heart rate signal from said sensor for measuring the heart rate signal;
analyzing said heart rate signal or the heart rate obtained from said heart rate signal by using a criterion to check the quality of the heart rate or the heart rate signal;
rejecting the heart rate or the heart rate signal when it is of low quality;
accepting the heart rate or the heart rate signal when it is not rejected;
using at least one activity sensor;
measuring the activity signal from said activity sensor;
calculating the relationship between said accepted heart rate signal or heart rate and said activity signal;
monitoring said calculated relationship between said accepted heart rate signal or heart rate and said activity signal;
modeling the heart rate or the heart rate signal as a function of the activity signal based on said calculated relationship; and
simulating the heart rate or the heart rate signal based on said calculated relationship when the heart rate or the heart rate signal is rejected.

19. The method according to claim 1, further comprising:
attaching at least one set of electrodes for monitoring heart rate to a body of the human or the animal;
measuring the heart rate signal from said set of electrodes;
analyzing said heart rate signal from said set of electrodes by comparing said heart rate signal with a set of reference values;
rejecting the heart rate signal when the heart rate signal deviates from said reference values;
accepting the heart rate signal when the heart rate signal is not rejected;
calculating the heart rate from an accepted heart rate signal;
attaching at least one activity sensor to the body;
measuring an activity signal from said activity sensor;
calculating a relationship between said heart rate or said accepted heart rate signal and said activity signal;
monitoring said relationship between said heart rate or said accepted heart rate signal and said activity signal;
modeling the heart rate or the heart rate signal as a function of the activity signal based on said calculated relationship; and
simulating the heart rate or the heart rate signal based on said calculated relationship when the heart rate signal is rejected.

20. A device for monitoring a heart rate of a human or an animal, according to claim 1, comprising:
a detection system with sensors for measuring the heart rate signal and the activity signal;
a computing unit for calculating the heart rate from the heart rate signal and for calculating the relationship between the activity signal and the heart rate or the heart rate signal;
a simulating unit for simulating the heart rate signal or the heart rate based on the relationship between the activity signal and the heart rate or the heart rate signal;
an evaluation unit for accepting the heart rate signal or the heart rate when the heart rate signal or the heart rate qualifies or for rejecting the heart rate signal or the heart rate when the heart rate signal or the heart rate is of low quality; and
an output unit for making available the measured and/or simulated heart rate or heart rate signal and/or the relationship between the activity signal and the heart rate signal or the heart rate.
